(19) United States
(12) Patent Application Publication
(10) Pub. No.: US 2004/0115483 A1
(43) Pub. Date: Jun. 17, 2004

(54) Crystal modification of a cyclic depsipeptide having improved strength

(76) Inventors: Jochen Kalbe, Leichlingen (DE); Michael Traubel, Koln (DE); Achim Harder, Koln (DE); Georg von Samson-Himmelstjerna, Mellendorf (DE)

Correspondence Address: Richard S Bullitt, Bayer Healthcare, 36 Columbia Turnpike, Morristown, NJ, 07962-1910, US

(21) Appl. No.: 10/470,853

(22) PCT Filed: Jan. 21, 2002
(86) PCT No.: PCT/EP02/00541

(30) Foreign Application Priority Data
Feb. 1, 2001 (DE) 10104362.7

(51) Int. Cl. B32B 9/00; B32B 19/00

(52) U.S. Cl. 428/698

(57) ABSTRACT

The invention relates to the use of crystal form (I) of the cyclic depsipeptide of the formula (I) for preparing medicaments, in particular for controlling endoparasites.

The invention relates to the use of crystal form I of the cyclic depsipeptide of the formula (I) for preparing medicaments, in particular for controlling endoparasites. The cyclic depsipeptide of the formula (I) is already known from EP-A-0 634 408 (WO 93/19053).

It is also known that this active compound exists in four different crystal forms, as described in EP-A-1 031 565 (WO 99/24412). The form described therein as crystal (V) has already been disclosed in EP-A-0 872 481 (WO 97/02256), together with a novel preparation process.

In the present application, the following names are used for the different forms: In EP-A-1 031 565, form I is referred to as “Crystal (III)” and has a melting point of 191.9° C.

In EP-A-1 031 565 form II is referred to as “Crystal (II)” and has a melting point of 182.4° C.

In EP-A-1 031 565, form m is referred to as “Crystal (1)” and has a melting point of 157.8° C.

In EP-A-1 031 565, form IV is described as “Prior Art Crystal (V)” and has a melting point of 145.0° C.;

according to what is said in EP-A-1 031 565, this form was already known from EP-A-0 872-481.

Of the four forms, form I is the thermodynamically most stable form, form II is the thermodynamically second most stable form, form II is the thermodynamically third most stable form and form IV is the thermodynamically fourth most stable form.

Surprisingly, it has now been found that among the forms mentioned, the thermodynamically most stable form I has the greatest bioavailability and therefore the greatest activity. For the person skilled in the art, this was unexpected: with this sparingly water-soluble active compound, solubility and bioavailability would be expected to decrease with increasing thermodynamic stability.

The invention relates to medicaments comprising the depsipeptide of the formula (I)

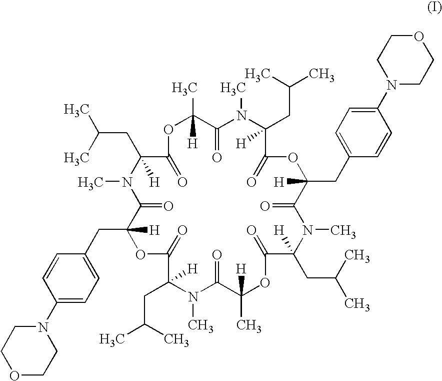

as a solid in crystal form I.

Owing to the superior bioavailability of form I, the medicament in question should contain as high a proportion as possible of this form. Thus, in the medicament in question, preferably at least 50%, particularly preferably at least 80%, very particularly preferably at least 90%, of the active compound of the formula I should be present in crystal form I. Ideally, in the medicament the depsipeptide is substantially completely present in form I (i.e. in a proportion of more than 99%).

According to the invention, the medicaments comprising the compound of the formula (I) in form I can be used for controlling pathogenic endoparasites encountered in humans and in animal husbandry and livestock breeding, in productive livestock, breeding stock, zoo animals, laboratory animals, animals used in experiments, and pets. They are active against resistant and normally sensitive species and against all or some stages of development of the pests. By controlling the pathogenic endoparasites, it is intended to reduce disease, mortality and decreasing performance (for example in the production of meat, milk, wool, hides, eggs, honey, etc.), so that more economical and simpler animal keeping is possible by using the active compounds. The medicaments are preferably used to control pathogenie endoparasites in warm-blooded animals. The pathogenic endoparasites include Cestodes, Trematodes; Nematodes and Acantocephales, in particular:

From the order of the Pseudophyllidea, for example Diphyllobothrium spp., Spirometra spp., Schistocephalus spp., Ligula spp., Bothridium spp., Diphlogonoporus spp.

From the order of the Cyclophyllidea, for example Mesocestoides spp., Anoplocephala spp., Paranoplocephala spp., Moniezia spp., Thysanosomsa spp., Thysaniezia spp., Avitellina spp., Stilesia spp., Cittotaenia spp., Andyra spp., Bertiella spp., Taenia spp., Echinococcus spp., Hydatigera spp., Davainea spp., Raillietina spp., Hymenolepis spp., Echinolepis spp., Echinocotyle spp., Diorchis spp., Dipylidium spp., Joyeuxiella spp., Diplopylidium spp.

From the subclass of the Monogenea, for example Gyrodactylus spp., Dactylogyrus spp., Polystoma spp.

From the subclass of the Digenea, for example Diplostomum spp., Posthodiplostomum spp., Schistosoma spp., Trichobilharzia spp., Omithobilharzia spp., Austrobilharzia spp., Gigantobilharzia spp., Leucochloridium spp., Brachylaima spp., Echinostoma spp., Echinoparyphium spp., Echinochasmus spp., Hypoderaeum spp., Fasciola spp., Fasciolides spp., Fasciolopsis spp., Cyclocoelum spp., Typhlocoelum spp., Paramphistomum spp., Calicophoron spp, Cotylophoron spp., Gigantocotyle spp., Fischoederius spp., Gastrothylacus spp., Notocotylus spp., Catatropis spp., Plagiorchis spp., Prosthogonimus spp., Dicrocoelium spp., Eurytrema spp., Troglotrema spp., Paragonimus spp., Collyriclum spp., Nanophyetus spp., Opisthorchis spp., Clonorchis spp., Metorchis spp., Heterophyes spp., Metagonimus spp.

From the order of the Enoplida, for example Trichuris spp., Capillaria spp., Trichom-osoides spp., Trichinella spp.

From the order of the Rhabditida, for example Micronema spp., Strongyloides spp.

From the order of the Strongylida, for example Stronylus spp., Triodontophorus spp., Oesophagodontus spp., Trichonema spp., Gyalocephalus spp., Cylindropharynx spp., Poteriostomum spp., Cyclococercus spp., Cylicostephanus spp., Qesophagostomum spp., Chabertia spp., Stephanurus spp., Ancylostoma spp., Uncinaria spp., Bunostom-um spp., Globocephalus spp., Syngamus spp., Cyathostoma spp., Metastrongylus spp., Dictyocaulus spp., Muellerius spp., Protostrongylus spp., Neostrongylus spp., Cystocaulus spp., Pneumostrongylus spp., Spicocaulus spp., Elaphostrongylus spp., Parelaphostrongylus spp., Crenosoma spp., Paracrenosoma spp., Angiostrongylus spp., Aelurostrongylus spp., Filaroides spp., Parafilaroides spp., Trichostrongylus spp., Haemonchus spp., Ostertagia spp., Marshallagia spp., Cooperia spp., Nematodirus spp., Hyostrongylus spp., Obeliscoides spp., Amidostomum spp., Ollulanus spp.

From the order of the Oxyurida, for example Oxyuris spp., Enterobius spp., Passalur-us spp., Syphacia spp., Aspiculuris spp., Heterakis spp.

From the order of the Ascaridia, for example Ascaris spp., Toxascaris spp., Toxocara spp., Parascaris spp., Anisakis spp., Ascaridia spp.

From the order of the Spirurida, for example Gnathostoma spp., Physaloptera spp., Thelazia spp., Gongylonema spp., Habronema spp., Parabronema spp., Draschia spp., Dracunculus spp.

From the order of the Filariida, for example Stephanofilaria spp., Parafilaria spp., Setaria spp., Loa spp., Dirofilaria spp., Litomosoides spp., Brugia spp., Wuchereria spp., Onchocerca spp.

From the order of the Gigantorhynchida, for example Filicollis spp., Moniliformis spp., Macracanthorhynchus spp., Prosthenorchis spp.

The livestock and breeding stock include mammals, such as, for example, cattle, horses, sheep, pigs, goats, camels, water buffalo, donkeys, rabbits, fallow deer, reindeer, fur-bearing animals, such as, for example, mink, chinchilla or racoon, birds, such as, for example chickens, geese, turkeys or ducks, freshwater fish and sea fish, such as, for example, trout, carp and eels, reptiles and insects, such as, for example, honey bee and silkworm.

The laboratory and test animals include mice, rats, guinea pigs, golden hamsters, dogs and cats.

The pets include dogs and cats.

Administration can be effected prophylactically as well as therapeutically.

According to the invention, suitable pharmaceutidal formulations are naturally only those in which the active compound is present as a solid in crystl form I. The advantages described are not observed in formulations in which the active compounds is present exclusively in dissolved or amorphous form.

The active substances are administered, either directly or in the form of suitable preparations, enterally, parenterally, dermally, nasally, by treating the habitat or with the aid of shaped articles containing the active compound, such as, for example, strips, plates, tapes, collars, ear tags, limb bands or marking devices.

Enteral administration of the active compounds is effected, for example, orally in the form of powders, tablets, capsules, pastes, drinks, granules, suspensions, boluses, medicated feed or drinking water. Dermal application is effected, for example, in the form of dipping, spraying, or pouring-on and spotting-on. Parenteral administration is effected, for example, in the form of injection (intramuscular, subcutaneous, intravenous or intraperitoneal) or by implants.

Suitable preparations include:

pour-on formulations, gels;

suspensions for oral or dermal administration and for injection; semi-solid preparations;

formulations in which the active compound is incorporated in a ointment base or in an oil-in-water or water-in-oil emulsion base;

solid preparations, such as powders, premixes or concentrates, granules, pellets, tablets, boluses, capsules; aerosols and inhalants, shaped articles containing the active compound.

Pour-on and spot-on formulations are poured or splashed onto limited areas of the skin, the active compound penetrating the skin and acting systemically.

Pour-on and spot-on formulations are prepared by suspending the active compound in suitable liquid auxiliaries or mixtures which are tolerated by the skin. If appropriate, other auxiliaries, such as colorants, absorption promoters, antioxidants, photostabilizers or tackifiers are added.

Suitable liquid auxiliaries include: water, alkanols, glycols, polyethylene glycols, propylene glycol, polypropylene glycols, glycerol, sorbitol, phenoxyethanol, esters, such as ethyl acetate, ethers, such as alkylene glycol alkyl ethers, such as dipropylene glycol monomethyl ether or diethylene glycol monobutyl ether, aromatic and/or aliphatic hydrocarbons, vegetable or synthetic oils, 2,2-dimethyl-4-oxymethylene-1,3-dioxolane.

Mention may also be made of: Paraffin oils, silicone oils, natural vegetable oils such as sesame seed oil, almond oil or castor oil, synthetic triglycerides, such as caprylic/capric acid biglyceride, a triglyceride mixture with vegetable fatty acids of chain length C₈₋₁₂ or other specifically selected natural fatty acids, mixtures of partial glycerides of saturated or unsaturated fatty acids which may also contain hydrbxyl groups, and mono- and diglycerides of the C₈/C₁₀-fatty acids.

Fatty acid esters, such as ethyl stearate, di-n-butyryl adipate, hexyl laurate, dipropylene glycol pelargonate, esters of a branched fatty acid having a medium chain length with saturated fatty alcohols of chain length C₁₆-C₁₈, isopropyl myristate, isopropyl palmitate, caprylic/capric esters of saturated fatty alcohols of chain length C₁₂-C₁₈, isopropyl stearate, oleyl oleate, decyl oleate, ethyl oleate, ethyl lactate, waxy fatty acid esters such as artificial duck uropygial fat, dibutyl phthalate, diisopropyl adipate, ester mixtures related to the latter, etc.

Fatty alcohols, such as isotridecyl alcohol, 2-octyldodecanol, cetylstearyl alcohol or oleyl alcohol.

Fatty acids, such as, for example, oleic acid and its mixtures.

Coloranis are all colorants which can be dissolved or suspended and which are approved for use in animals.

Examples of absorption promoters are DMSO, spreading oils, such as isopropyl myristate, dipropylene glycol pelargonate, silicone oils, fatty acid esters, triglycerides or fatty alcohols.

The following are antioxidants: sulfites or metabisulfites, such as potassium metabisulfite, ascorbic acid, butylhydroxytoluene, butylhydroxyanisole, tocopherol or propyl gallate.

Example of photostabilizers are novantisolic acid.

Tackifiers are, for example, cellulose derivatives, starch derivatives, polyacrylates or natural polymers such as alginates or gelatin.

Suitable other auxiliaries include: substances which increase the viscosity and stabilize the suspension, such as carboxymethylcellulose, methylcellulose and other cellulose and starch derivatives, polyacrylates, alginates, gelatin, gum arabic, polyvinylpyrrolidone, polyvinyl alcohol, methyl vinyl ether/maleic anhydride copolymers, polyethylene glycols, waxes, colloidal silica, or mixtures of the listed substances.

Suspensions can be administered orally, dermally or as an injection. They are prepared by suspending the active compound in a liquid excipient, if appropriate with the addition of other auxiliaries, such as wetting agents, colorants, absorption promoters, preservatives, antioxidants and photostabilizers.

Suitable wetting agents (dispersants) are:

nonionic surfactants, for example polyethoxylated castor oil, polyethoxylated sorbitan monooleate, sorbitan monostearate, glycerol monostearate, polyoxyethyl stearate or alkylphenol polyglycol ethers;

ampholytic surfactants, such as disodium N-lauryl-β-iminodipropionate or lecithin;

anionic surfactants, such as sodium lauryl sulfate, fatty alcohol ether sulfates, the monoethynolamine salt of mono/dialkylpolyglycol ether orthophosphoric ester.

Suitable other auxiliaries include those indicated further above.

Semi-solid preparations can be administered orally or dermally. They are only distinguished from the above-described suspensions and emulsions by their higher viscosity.

To prepare solid preparations, the active compound is mixed with suitable excipients, if appropriate with the addition of auxiliaries, and the mixture is formulated as desired.

Suitable excipients include all physiologically acceptable solid inert substances. Suitable for this purpose are inorganic and organic substances. Inorganic substances are, for example, sodium chloride, carbonates, such as calcium carbonate, hydrogen carbonates, aluminum oxides, silicas, clays, precipitated or colloidal silica, and phosphates.

Organic substances are, for example, sugar, cellulose, foodstuffs and animal feeds, such as powdered milk, animal meals, cereal meals, coarse cereal meals and starches.

Auxiliaries are preservatives, antioxidants and colorants which have already been mentioned further above.

Other suitable auxiliaries are lubricants and glidants, such as, for example, magnesium stearate, stearic acid, talc, bentonites, disintegrants, such as starch or crosslinked polyvinylpyrrolidone, binders, such as, for example, starch, gelatin or linear polyvinylpyrrolidone, and dry binders, such as microcrystalline cellulose.

In the preparations, the active compounds can also be present in mixtures with synergists or other active compounds which are active against pathogenic endoparasites. Examples of such active compounds are L-2,3,5,6-tetrahydro-6-phenylimidazothiazole, benzimidazole carbamates, praziquantel, pyrantel or febantel.

Ready-to-use preparations contain the active compound in concentrations of from 10 ppm to 20 percent by weight, preferably from 0.1 to 10 percent by weight.

Preparations which are diluted before use contain the active compound in concentrations of 0.5-90% by weight, preferably 5-50% by weight.

In general, it has been found to be advantageous to administer amounts of from about 0.1 to about 100 mg of active compound per kg of body weight per day to obtain effective results.

### EXAMPLES

### Example 1

### Effectiveness of Forms I-IV of the Depsipeptide of the Formula (I) at Different Oral Dosages Against Haemonchus Contortus in Sheep

Sheep (Merino or Blackhead breed, 25-35 kg of body weight) were experimentally infected with 5000H contortus L3 larvae and treated with the formulated test substance at the end of the prepatency time of the parasite. The test compounds were administered orally (gelatin capsule). The anthelmintic effectiveness of the test substances was measured as a function of reduction of the number of eggs per gram of faeces. To this end, fresh faeces from the test animals were processed according to the McMaster method, modified according to Wetzel, and the number of eggs was determined. The number of eggs was determined at regular intervals before and after the treatment. The anthelmintic effectiveness was defined as follows: 3=>95%, 2=75-95%, 1=50-75% and 0=<50% reduction in eggs (cf. also G. von Samson-Himmelstjerna, A. Harder, T. Schnieder, J. Kalbe, N. Mencke (2000) In vivo activities of the new anthelmintic depsipeptide PF1022A. Parasitol. Res. 86:194-199).

| | | Dosage | |
|---|---|---|---|
| Nematode | Form | (mg/kg) | Effectiveness |
| H. contortus | I | 1.0 | 3 (1 Sheep, EPG) |
| H. contortus | I | 0.5 | 3 (1 Sheep, EPG) |
| H. contortus | I | 0.1 | 3 (1 Sheep, EPG) |
| H. contortus | II | 1.0 | 3 (1 Sheep, EPG) |
| H. contortus | II | 0.5 | 0 (1 Sheep, EPG) |
| H. contortus | II | 0.1 | 0 (1 Sheep, EPG) |
| H. contortus | III | 1.0 | 3 (1 Sheep, EPG) |
| H. contortus | III | 0.5 | 1 (1 Sheep, EPG) |
| H. contortus | III | 0.1 | 1 (1 Sheep, EPG) |
| H. contortus | IV | 1.0 | 3 (1 Sheep, EPG) |
| H. contortus | IV | 0.5 | 0 (1 Sheep, EPG) |
| H. contortus | IV | 0.1 | 0 (1 Sheep, EPG) |

3 = effectiveness > 95%;
2 = effectiveness 75% to 95%;
1 = effectiveness 50% to 75%;
0 = effectiveness ≦ 50 %;
EPG = eggs per gram of faeces in the egg reduction test.

### Example 2

### Effectiveness of Forms I and IV of the Depsipeptide of the Formula (I) at Different Oral Dosages Against Cooperia Oncophora in Cattle

Cattle (breed: Hostein Friesean, species: Bos taurus, calves or young cattle of up to about 200 kg) were experimentally infected with 15,000 C. oncophora L3 larvae. After successful infection (prepatency time about 18-21 days) the number of eggs per gram of faeces was determined 3 times within one week. The animals were then (at day 0=day of the treatment) treated orally (gelatin capsule) or subcutaneously with the formulated test substance. The anthelmintic effectiveness of the test substances was determined as a function of the reduction of the number of worms (in % of the untreated control) in the small intestine after the animals had been slaughtered (10 days after the treatment) (cf. also G. von Samson-Himmelstjerna, A. Harder, T. Schnieder, J. Kalbe, N. Mencke (2000) In vivo activities of the new anthelmintic depsipeptide PF1022A. Parasitol. Res. 86: 194-199.).

| | | Dosage | |
|---|---|---|---|
| Nematode | Form | (mg/kg) | Effectiveness |
| C. oncophora | I | 1.0 | 93.81% (3 cattle, slaughter test) |
| C. oncophora | IV | 2.0 | 99.15% (3 cattle, slaughter test) |
| C. oncophora | IV | 1.0 | 43.09% (3 cattle, slaughter test) |

The effectiveness in the slaughter experiment is stated in % reduction in worms, compared to the untreated control.

1. A medicament, comprising the depsipeptide of the formula (I) 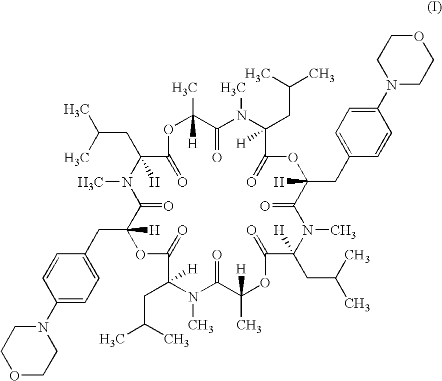
as a solid in crystal form I.

2. A medicament as claimed in claim 1 where at least 50% of the depsipeptide of the formula (I) is present in crystal form I.

3. A medicament as claimed in claim 1 where at least 80% of the depsipeptide of the formula (I) is present in crystal form I.

4. A medicament as claimed in claim 1 where at least 90% of the depsipeptide of the formula (I) is present in criptal form I.

5. A medicament as claimed in claim 1 where at least 99% of the depsipeptide of the formula (I) is present in crystal form I.

6. The use of the depsipeptide of the formula (I) 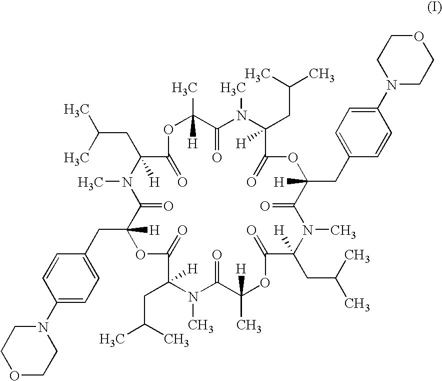
as a solid in crystal form I for preparing medicaments comprising the depsipeptide of the formula (I) in crystal form I.

7. The use as claimed in claim 6 for preparing oral medicaments for animals.

8. The use as claimed in claim 6 for controlling endoparasites, in particular Cestodes, Trematodes, Nematodes, Acantocephala in animals.

9. A method for controlling endoparasites in animals, comprising a step where an effective amount of the depsipeptide of the formula (I) 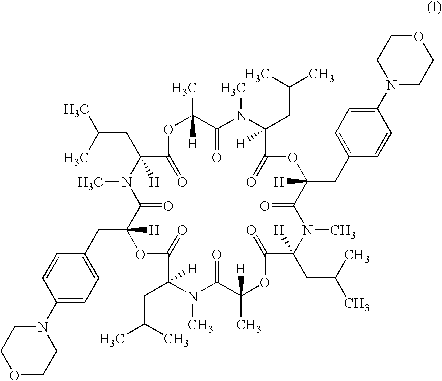
in crystal form I is administered to the animal.
